(19) United States
(12) Patent Application Publication
(10) Pub. No.: US 2019/0085405 A1
(43) Pub. Date: Mar. 21, 2019

(54) METHODS OF DETECTING APOBEC3 EXPRESSION AND PREDICTING CLINICAL OUTCOMES

(71) Applicants: REGENTS OF THE UNIVERSITY OF MINNESOTA, Minneapolis, MN (US); MAYO FOUNDATION FOR MEDICAL EDUCATION AND RESEARCH, Rochester, MN (US)

(72) Inventors: Reuben S. Harris, St. Paul, MN (US); Brandon C. Leonard, San Francisco, CA (US); Scott H. Kaufmann, Rochester, MN (US)

(21) Appl. No.: 16/086,985

(22) PCT Filed: Mar. 23, 2017
(86) PCT No.: PCT/US2017/023783

§ 371 Date: Sep. 20, 2018

Related U.S. Application Data
(60) Provisional application No. 62/312,115, filed on Mar. 23, 2016.

(51) Int. Cl. C12Q 1/6886; C07K 16/18; C07K 16/08; C07K 16/40; C12Q 1/6853

(52) U.S. Cl. CPC C12Q 1/6886; C07K 16/18; C07K 16/087; C12Q 2600/118; C12Q 1/6853; C12Q 2600/158; C07K 16/40

(57) ABSTRACT

This disclosure describes a kit for determining the level of APOBEC3 in a sample of a subject, and a method for making an anti-APOBEC3 antibody/APOBEC3 complex. This disclosure further describes methods of determining a prognosis of a subject having a cancer using APOBEC3 expression and, in some instances, providing treatment based on the prognosis.

### CROSS-REFERENCE TO RELATED APPLICATION

This application claims priority to U.S. Provisional Patent Application No. 62/312,115, filed Mar. 23, 2016, which is incorporated herein by reference.

### GOVERNMENT FUNDING

This invention was made with government support under CA136393 awarded by the National Institutes of Health. The government has certain rights in the invention.

### SEQUENCE LISTING

This application contains a Sequence Listing electronically submitted to the United States Patent and Trademark Office via EFS-Web as an ASCII text file entitled “2017-03-21-SequenceListing_ST25.txt” having a size of 5 KB and created on 21 Mar. 2017. Due to the electronic filing of the Sequence Listing, the electronically submitted Sequence Listing serves as both the paper copy required by 37 CFR § 1.821(c) and the CRF required by § 1.821(e). The information contained in the Sequence Listing is incorporated by reference herein.

### SUMMARY OF THE INVENTION

In one aspect, this disclosure describes a method that includes: detecting a level of APOBEC3 expression in a sample form a subject; and determining the prognosis of the subject based upon the level of APOBEC3 expression. In some embodiments, the method further includes providing treatment to the subject based upon the prognosis.

In another aspect, this disclosure a method for making an anti-APOBEC3 antibody/APOBEC3 complex. The method includes: providing a sample including APOBEC3; and contacting the sample with an anti-APOBEC3 antibody under conditions that permit the binding of the anti-APOBEC3 antibody to APOBEC3 to yield the anti-APOBEC3 antibody/APOBEC3 complex. In some embodiments, the sample can be from a subject having a cancer or a tumor.

In a further aspect, this disclosure describes a kit for determining a level of APOBEC3 in a sample from a subject. The kit includes reagents for determining the level of expression of APOBEC3 in the sample; and instructions for how to use the reagents.

In another aspect, this disclosure describes a method including detecting APOBEC3 in a sample from a subject and detecting the level of expression of a T cell marker in the sample.

The words “preferred” and “preferably” refer to embodiments of the invention that may afford certain benefits, under certain circumstances. Other embodiments may also be preferred, however, under the same or other circumstances. Furthermore, the recitation of one or more preferred embodiments does not imply that other embodiments are not useful, and is not intended to exclude other embodiments from the scope of the invention.

The terms “comprises” and variations thereof do not have a limiting meaning where these terms appear in the description and claims.

Unless otherwise specified, “a,” “an,” “the,” and “at least one” are used interchangeably and mean one or more than one.

Also herein, the recitations of numerical ranges by endpoints include all numbers subsumed within that range (e.g., 1 to 5 includes 1, 1.5, 2, 2.75, 3, 3.80, 4, 5, etc.).

For any method disclosed herein that includes discrete steps, the steps may be conducted in any feasible order. And, as appropriate, any combination of two or more steps may be conducted simultaneously.

The above summary of the present invention is not intended to describe each disclosed embodiment or every implementation of the present invention. The description that follows more particularly exemplifies illustrative embodiments. In several places throughout the application, guidance is provided through lists of examples, which examples can be used in various combinations. In each instance, the recited list serves only as a representative group and should not be interpreted as an exclusive list.

### BRIEF DESCRIPTION OF THE FIGURES

FIG. 1 shows correlations between APOBEC3 expression and T cell markers in high-grade serous ovarian carcinoma (HGSOC). Dot plots illustrating correlations between APOBEC3G (panels A-F) or APOBEC3B (panels G-L) expression and the indicated T cell marker (n=354). mRNA expression was determined using quantitative reverse transcription polymerase chain reaction (RT-qPCR) and normalized to the housekeeping gene TBP. Spearman's correlation coefficients (rₛ) and p-values are shown. Best-fit lines are shown for qualitative comparison, and were calculated using linear regression models.

FIG. 2 shows immunohistochemistry and immunofluorescence of T cell markers in HGSOC. Photomicrographs of immunohistochemistry and immunofluorescence staining performed on HGSOC specimens illustrating the association between levels of T lymphocyte infiltration and the intensity of APOBEC3G expression. Representative staining of one HGSOC specimen with low (patient 6) and three staining sets from two HGSOC specimens with high (patients 3 and 2) levels of T cell infiltration are shown. The images depict hematoxylin (panels A, F, K, and P), CD3 (panels B, G, L, and Q), CD4 (panels C, H, M, and R), CD8 (panels D, I, N, and S), and APOBEC3G (panels E, J, O, and T). The dotted box in the 40× hematoxylin images indicates the approximate location of the subsequent panels at 100× magnification. The bottom row shows representative 1000× magnification images of colocalization of CD3 and APOBEC3G by immunofluorescent staining. DAPI-stained nuclei are blue.

FIG. 3 shows clinical correlates of T cell marker expression in HGSOC. Kaplan-Meier plots illustrating associations between progression free survival (PFS) (panel A, n=354) or overall survival (OS) (panel B, n=348) and either one of the conventional T cell markers or APOBEC3G or APOBEC3B expression in the Mayo cohort of patients. Samples were split at the median expression level for each gene with one line of each plot representing tumors with high mRNA levels and the other line of each plot representing tumors with low mRNA levels.

FIG. 4 shows correlations between APOBEC expression and immune cell markers across 22 cancer types. Heatmap of Spearman's correlation coefficients calculated from the comparison of the T cell marker CD3D (top panel) or the B cell marker CD20 (bottom panel) with the indicated APOBEC family member. Expression levels were determined using TCGA RNAseq data (see Table 5 for the long form for each tumor abbreviation).

FIG. 5 shows validation of RT-qPCR assays. (Panel A) Histograms reporting mRNA levels of CD3D, CD4, and CD8A in peripheral blood mononuclear cells and CD4+ T cells isolated from the same donor. mRNA levels were quantified by RT-qPCR and represent the average of triplicate reactions. Error bars denote standard deviation (SD). (Panel B) Histograms reporting surface expression of CD3δ, CD4, and CD8α in peripheral blood mononuclear cells and CD4+ T cells isolated from the same donor. Each bar represents the percent of live cells that are positive for each marker as quantified by flow cytometry.

FIG. 6 shows clinical correlates of APOBEC3G expression in two independent cohorts of HGSOC. Samples were split at the median APOBEC3G expression value with the light gray line representing tumors with high mRNA levels and the dark gray line representing tumors with low mRNA levels, as indicated. Statistical values were calculated using the Mantel-Cox log-rank test. (Panel A) Kaplan-Meier plot of progression free survival (PFS) in the KM plotter cohort. (Panel B) Kaplan-Meier plot of overall survival (OS) in the KM plotter cohort. (Panel C) Kaplan-Meier plot of progression free survival (PFS) in the Dutch cohort. (Panel D) Kaplan-Meier plot of overall survival (OS) in the Dutch cohort.

### DETAILED DESCRIPTION OF ILLUSTRATIVE EMBODIMENTS

Many cancers including, for example, ovarian cancer, continue to be deadly, in some cases because of lack of clinical detection or the ability to accurately assess prognosis. Better methods for detection and the determination of patient outcomes are needed. This disclosure describes compositions, kits, and methods of using APOBEC3 expression as a predictive biomarker for anti-cancer responses and clinical outcomes. In some aspects, this disclosure describes a method of determining a prognosis of a subject having a cancer. In some cases, the subject may be treated based on the prognosis. The disclosure further describes a kit for determining the level of APOBEC3 in a sample of a subject, and a method for making an anti-APOBEC3 antibody/APOBEC3 complex.

Ovarian cancer is the deadliest gynecological malignancy worldwide. The most common type of ovarian cancer, high-grade serous ovarian carcinoma (HGSOC), accounts for over 60% of cases, and is the most aggressive reproductive track malignancy. Due to the lack of efficient detection methods, HGSOC generally presents at advanced stages and is associated with high rates of recurrence and mortality. Interestingly, T cell infiltration has been identified as a favorable prognostic factor for HGSOC (Knutson et al. Cancer Immunol Immunother. 2015; 64:1495-504; Nielsen et al. Clin Cancer Res. 2012; 18:3281-92; Zhang et al. N Engl J Med. 2003; 348:203-13; Sato et al. Proc Natl Acad Sci USA. 2005; 102:18538-43; Preston et al. PLoS ONE. 2013; 8:e80063.) Additional markers of T cell infiltration and particularly those reflecting high-quality anti-tumor responses are needed to fully realize the clinical impact of this finding.

The APOBEC enzymes are an 11-member family of zinc-coordinating enzymes that convert cytosines to uracils (C-to-U) in ssDNA. The APOBEC enzymes include APOBEC1 (isoform a, NP_001291495.1; isoform b, NP_005880.2), APOBEC2 (NP_006780.1), APOBEC3A (isoform a, NP_663745.1; isoform b, NP_001257335.1), APOBEC3B (NP_004891.4), APOBEC3C (NP_055323.2), APOBEC3D (NP_689639.2), APOBEC3F (NP_660341.2), APOBEC3G (isoform 1, NP_068594.1; isoform 2, NP_001336365.1; isoform 3, NP_001336366.1; isoform 4, NP_001336367.1), APOBEC3H (isoform SV182, NP_001159474.2; isoform SV183, NP_861438.3; isoform SV200, NP_001159475.2; isoform SV154, NP_001159476.2), APOBEC4 (NP_982279.1), and activation-induced deaminase (AID/AICDA; isoform 1, NP_065712.1; isoform 2, NP_001317272.1). The enzymatic activity of specific family members including, for example, AID, is essential for both adaptive and innate immune responses. AID plays a role in antibody diversification through somatic hypermutation and class switch recombination in B cells. Other well-studied family members include APOBEC3D, APOBEC3F, APOBEC3G, and APOBEC3H. These enzymes are capable of introducing C-to-U lesions in viral cDNA intermediates that manifest as G-to-A mutations in proviral genomes. APOBEC3 subfamily members have also been implicated in restricting the replication of many other DNA-based parasites including transposable elements. Most APOBEC family members are expressed broadly and constitutively, but several family members can also be further upregulated by specific conditions. For example, APOBEC3A can be upregulated by interferon-α; APOBEC3B can be upregulated by non-canonical NF-κB activation and HPV infection; and APOBEC3C, APOBEC3D, APOBEC3F, APOBEC3G, and APOBEC3H can be upregulated by the combination of T cell activation and HIV-1 infection.

While the APOBEC3 enzymes have been shown to have physiologic roles in protecting cells from endogenous and exogenous DNA-based pathogens, their dysregulation has also been linked to cancer. For instance, AID has been linked to various B cell malignancies and APOBEC3B is overexpressed and a significant source of mutation in breast, ovarian, and several other cancers. APOBEC3B deaminates cytosines in genomic DNA to produce promutagenic uracil lesions, which can result in mutations if they are not repaired faithfully. APOBEC3B is overexpressed and its mutation signature is overrepresented in genomic sequences of HGSOC and other cancers. Elevated APOBEC3B expression levels have also been linked to poor prognosis in multiple cancer types. In addition to APOBEC3B, related subfamily members have also been implicated in cancer mutagenesis to varying degrees. Because most APOBEC family members are expressed in many normal cell types and tissues, a major confounding factor in quantification of APOBEC expression levels in tumors is cellular heterogeneity due to surrounding normal tissue and immune cell infiltration. To address these issues, the disclosure describes quantification of APOBEC expression in a large cohort of HGSOC patients and exploration of the correlation of APOBEC expression levels with immune cell infiltration. A strong positive correlation was identified between APOBEC3G expression and markers of T cell infiltration, and co-expression was confirmed by immunohistochemical and immunofluorescent staining of primary HGSOC specimens. Moreover, high APOBEC3G expression levels correlated significantly with improved prognosis. These findings were extended to multiple cancer types through an analysis of publically available RNA sequencing (RNAseq) data from The Cancer Genome Atlas (TCGA). Collectively, these results highlight the complexity of APOBEC3 family member expression in HGSOC specimens comprised of tumor cells, surrounding normal tissues, and in many instances infiltrating immune cells. This disclosure identifies APOBEC3G as a new candidate biomarker for effective T cell responses and potentially for immunotherapies against HGSOC.

This disclosure describes the unanticipated association between high levels of APOBEC3G expression and cytotoxic T lymphocyte (CTL) activation in HGSOC. This disclosure further describes the surprising identification of APOBEC3G as a new candidate biomarker for activated T lymphocyte infiltration in HGSOC and improved patient outcomes. This role for APOBEC3G is unexpected in part because prior work has shown that APOBEC3G is expressed broadly, constitutively, and in some instances inducibly (Refsland et al. Nucleic Acids Res. 2010; 38:4274-8; Hultquist et al. Journal of Virology. 2011; 85:11220-34; Koning et al. Journal of Virology. 2009; 83:9474-85; Liddament et al. Curr Biol. 2004; 14:1385-91; Burns et al. Nature. 2013; 494:366-70).

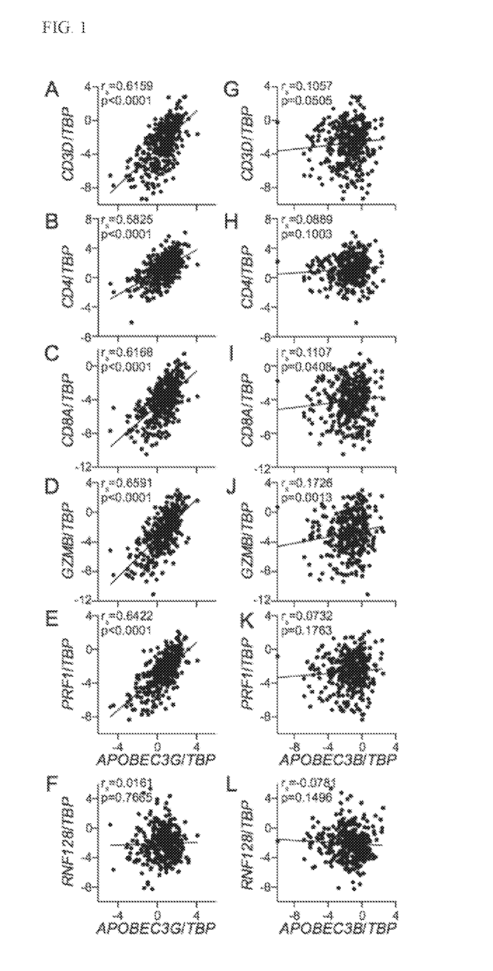

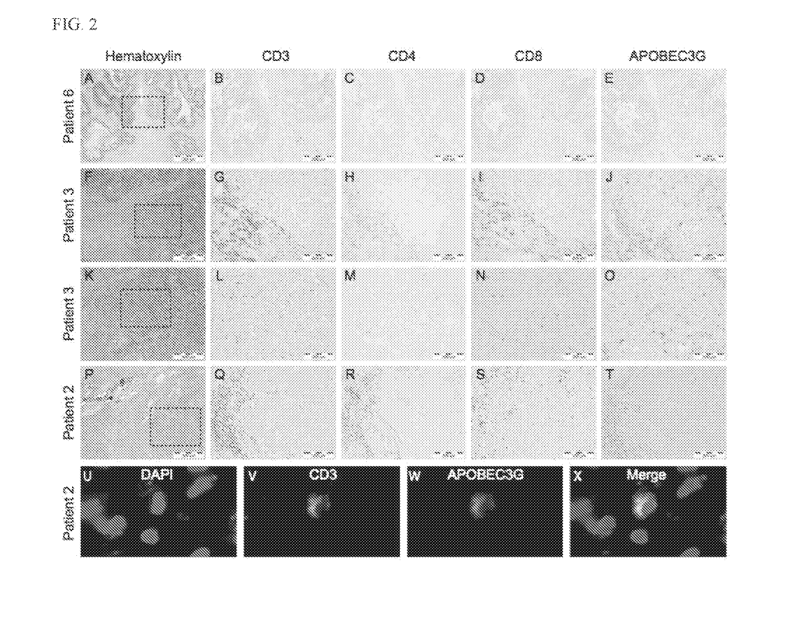

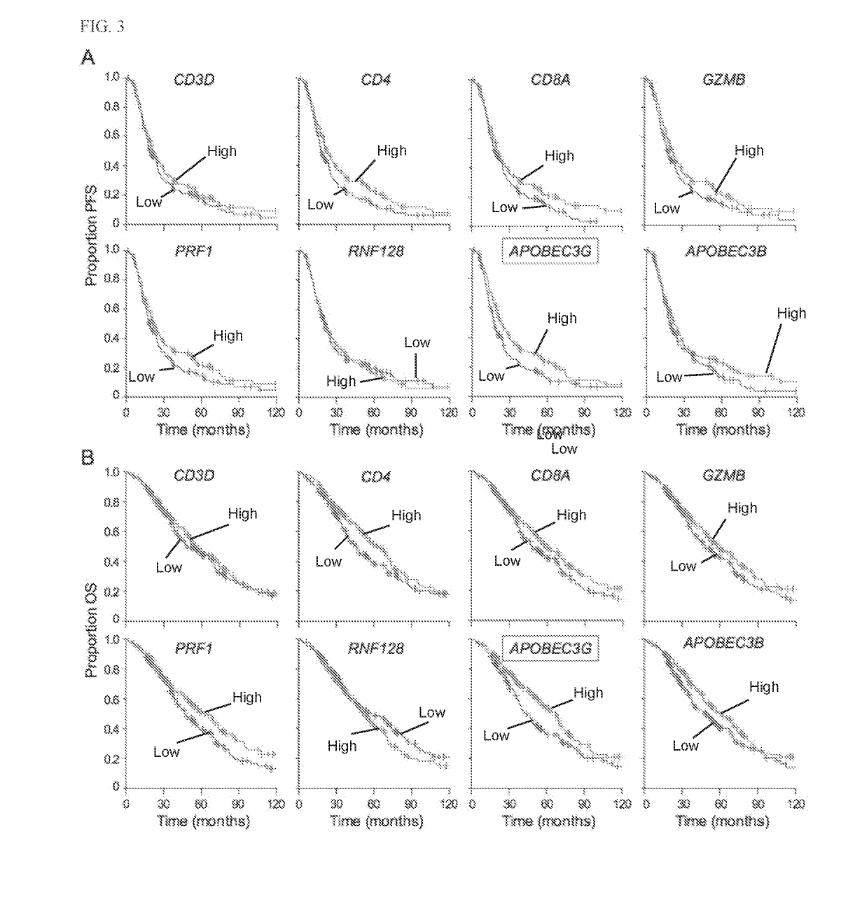

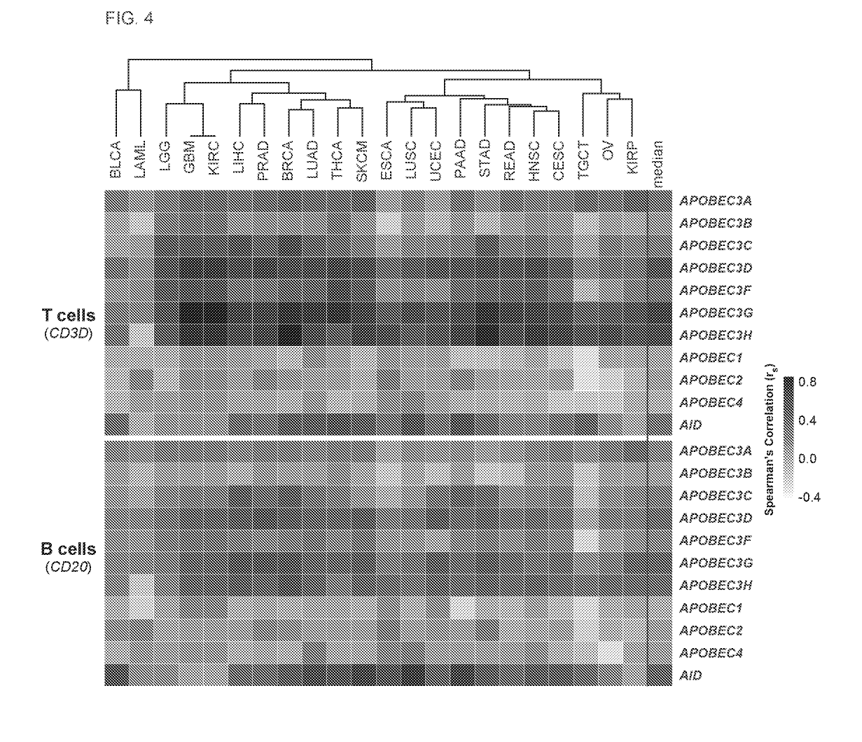

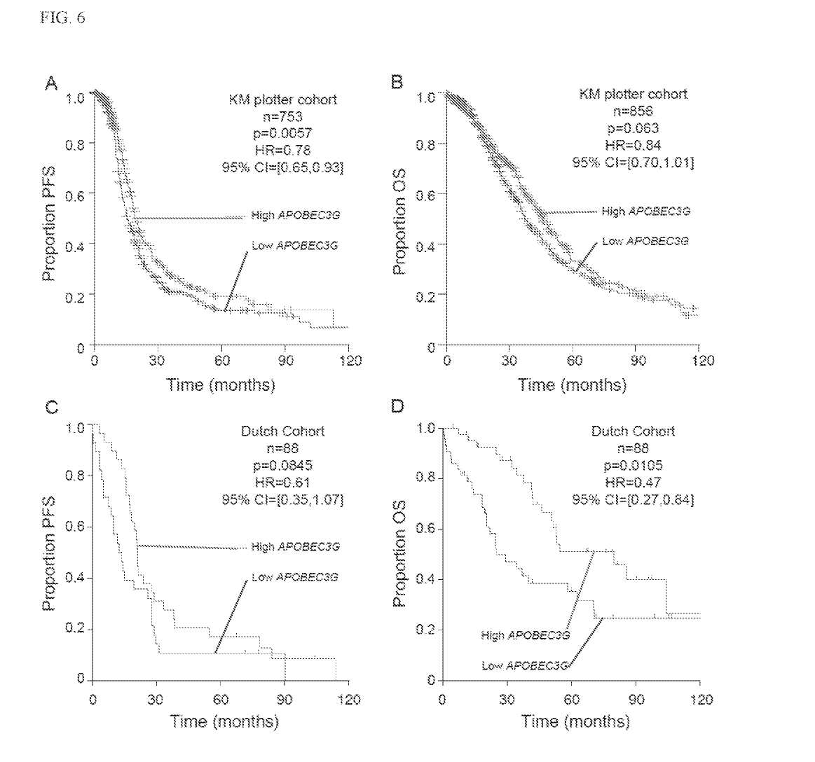

The analysis of a cohort of 354 HGSOC patients also identified a strong correlation between APOBEC3G and several markers of T cell infiltration (FIG. 1A-E). These results were validated at the protein level by immunohistochemistry and immunofluorescent staining of independent HGSOC tumor samples (representative image sets in FIG. 2). Clinical data revealed that APOBEC3G also associates with improved outcomes in a large HGSOC cohort as well as in two additional independent ovarian cancer cohorts (Table 2, FIG. 3, and FIG. 6). Finally, a global analysis of gene expression in 22 cancer types identified a similar correlation for two additional APOBEC3 genes, APOBEC3D and APOBEC3H, and a marker of T cells, CD3D (FIG. 4). Together, these data suggest that APOBEC3D, APOBEC3G, and APOBEC3H expression levels in tumor infiltrating T lymphocytes may be a predictive biomarker for strong anti-cancer T cell responses and improved HGSOC outcomes.

Over the past several years, substantial evidence has accumulated to indicate that APOBEC3B and AID contribute to cancer genome mutagenesis. APOBEC3B is thought to mutate the genome of several different cancer types, including breast, lung, bladder, cervical, head/neck, and ovarian (Burns et al. Nature. 2013; 494:366-70; Leonard et al. Cancer Research. 2013; 73:7222-31; Burns et al. Nat Genet. 2013; 45:977-83; Roberts et al. Nat Genet. 2013; 45:970-6; Alexandrov et al. Nature. 2013; 500:415-21). The carcinogenic effect of AID appears more limited, as its characteristic deamination signature has only been found in certain types of B cell leukemias and lymphomas (Robbiani et al. Annu Rev Pathol. 2013; 8:79-103; Alexandrov et al. Nature. 2013; 500:415-21). The idea that AID expression is constrained to B lineage cell types is consistent with data showing that AID mRNA expression correlates with CD20 mRNA expression in several solid tumor types (FIG. 4 bottom heatmap). In addition to APOBEC3B and AID, several other APOBEC family members have also been implicated in carcinogenesis. For example, APOBEC3G may drive hepatocellular carcinoma tumorigenesis. The idea that APOBEC3G drives tumorigenesis is, however, difficult to reconcile with the observation that APOBEC3G mRNA expression in HGSOC and other tumor types correlates with the expression of activated T lymphocyte markers. Moreover, at least for HGSOC, this strong correlation could be validated visually at the protein level (FIGS. 1,2, and 4).

Surprisingly, the analysis of APOBEC3B in high-grade serous ovarian carcinoma (HGSOC) revealed no significant clinical correlations and even a slight trend in the Mayo Clinic cohort toward better overall survival (FIG. 3). This finding differs significantly from estrogen receptor (ER)-positive breast cancer, where high APOBEC3B expression associates with shorter periods of disease-free survival and poorer rates of overall survival (Sieuwerts et al. Horm Cancer. 2014; 5:405-13; Cescon et al. Proceedings of the National Academy of Sciences. 2015; 112:2841-6; Periyasamy et al. Cell Rep. 2015; 13:108-21; Tsuboi et al. Breast Cancer. 2015; in press (October 17th Epub ahead of print)). A previous deep-sequencing study highlighting the similarities between HGSOC and another type of breast cancer, triple negative breast cancer (TNBC) (Cancer Genome Atlas Research Network. Nature. 2011; 474:609-15), also found no correlation between APOBEC3B expression and survival in TNBC (Cescon et al. Proceedings of the National Academy of Sciences. 2015; 112:2841-6). One major difference between these two cancer types (high-grade serous ovarian carcinoma and triple negative breast cancer) and estrogen receptor-positive breast cancer is therapeutic options. There are multiple targeted therapeutics available for the treatment of ER-positive breast cancer that are administered based on molecular markers. In contrast, nearly all HGSOC and many triple negative breast cancer patients are treated with platinum-based therapies. Because platinum-based therapies induce DNA damage, it is possible that these drugs become synergistic with APOBEC3B-catalyzed cytosine deamination and create a synthetic lethal state in cancer cells. This idea is reasonable because increased mutation loads have been shown to correlate with improved clinical outcomes in HGSOC patients treated with cisplatin (Sohn et al. Gynecol Oncol. 2012; 126:103-8). Furthermore, a synergistic effect created by these two forms of DNA damage could explain the slight trend found here toward a positive correlation between increased APOBEC3B expression and improved outcomes. Another possible explanation is that the levels of APOBEC3B mutagenesis in ovarian cancer may be not high enough to manifest clinically. Indeed, the APOBEC3B mutation signature is not as strong in ovarian cancer as it is in many other cancer types despite similar mRNA and protein expression levels. The underlying causes for this apparent discordancy are unknown, but several factors could be involved, including altered DNA repair capacities, differential protein regulation, and mutational contributions from other sources. More work is needed to determine the threshold of APOBEC3B mutagenesis needed to have a clinical impact.

Global analysis of TCGA data revealed that the correlation between APOBEC3F and CD3D was substantially diminished as compared to APOBEC3D, APOBEC3G, and APOBEC3H (FIG. 4). This result is surprising because APOBEC3F is thought to be broadly expressed and to play an equally important role as APOBEC3D, APOBEC3G, and APOBEC3H in HIV-1 restriction in CD4-positive T lymphocytes, and because APOBEC3F is expressed at comparable levels to APOBEC3D and at higher levels than APOBEC3H in human primary CD4+ T cells. The data in Example 1 suggest that APOBEC3F may be under- and/or heterogeneously-expressed in T cells associated with the tumor microenvironment. The data in Example 1 further indicate that much of the expression of several APOBEC family members in cancer is likely due to T and B cell infiltration.

In one aspect this disclosure describes a method of determining a prognosis of a subject having a cancer. In some embodiments, the method further includes treating the patient based on the prognosis. In some embodiments, the cancer is a primary tumor. In some embodiments, the cancer is metastatic (i.e., disseminated beyond the site of the primary tumor). In some embodiments, a cancer is a solid tumor. In some embodiments, the cancer is a blood cancer (for example, a leukemia or a lymphoma).

The cancer can involve any tissue or organ, such as bone, brain, breast, cervix, larynx, lung, pancreas, prostate, skin, spine, stomach, uterus, ovary, or blood. The cancer can be a bone cancer, brain cancer, breast cancer, cervical cancer, ovarian cancer, cancer of the larynx, lung cancer, pancreatic cancer, prostate cancer, skin cancer, cancer of the spine, stomach cancer, uterine cancer, or a blood cancer. The cancer can be a carcinoma. In some embodiments, the cancer includes one of the cancers listed in Table 5. In some embodiments, the cancer includes bladder urothelial carcinoma, acute myeloid leukemia, brain lower grade glioma, glioblastoma multiforme, kidney renal clear cell carcinoma, liver hepatocellular carcinoma, prostate adenocarcinoma, breast invasive carcinoma, lung adenocarcinoma, thyroid carcinoma, skin cutaneous melanoma, esophageal carcinoma, lung squamous cell carcinoma, uterine corpus endometrial carcinoma, pancreatic adenocarcinoma, stomach adenocarcinoma, testicular germ cell tumor, ovarian carcinoma, ovarian serous cystadenocarcinoma, kidney renal papillary cell carcinoma, or high-grade serious ovarian cancer (HGSOC).

In some embodiments, the method of determining a prognosis of the subject includes detecting the level of APOBEC3 expression in the sample from the subject; and determining the prognosis of the subject based upon the level of APOBEC3 expression. In some embodiments, the method can include obtaining a sample from the subject. In some embodiments, the sample is preferably derived from a tumor. APOBEC3 includes one or more of the APOBEC3 family members: APOBEC3A, APOBEC3B, APOBEC3C, APOBEC3DE, APOBEC3F, APOBEC3G, and APOBEC3H including each of the isoforms and splice variants thereof. In some embodiments, the APOBEC3 includes APOBEC3D, APOBEC3G, and/or APOBEC3H. In some embodiments, the APOBEC3 preferably includes APOBEC3 G.

In some embodiments, detecting the level of APOBEC3 expression includes detecting the level of expression of an RNA or a protein of one or more members of the APOBEC3 family. In some embodiments, detecting the level of APOBEC3 expression preferably includes detecting the RNA level of APOBEC3D, APOBEC3G, and/or APOBEC3H or detecting the protein level of APOBEC3D, APOBEC3G, and/or APOBEC3H. In some embodiments, detecting the level of APOBEC3 expression preferably includes detecting the RNA level of APOBEC3D, APOBEC3G, and/or APOBEC3H or detecting the protein level of APOBEC3D, APOBEC3G, and/or APOBEC3H.

In some embodiments the sample is a tissue sample including, for example, a tumor sample. The sample can be obtained from the subject using any suitable method. For example, a biopsy can be used to obtain the sample including, for example, a punch biopsy or a core needle biopsy. In some embodiments, the sample includes a cell derived from a tumor. A cell derived from a tumor can include a tumor cell and/or a tumor infiltrating cell including, for example, a lymphocyte. In some embodiments, the sample includes a tumor cell. In some embodiments, the sample includes a tumor-infiltrating cell including, for example, a lymphocyte and/or a T cell. In some embodiments, the sample includes a tissue section.

In some embodiments, the tissue sample includes a cell including at least one of the cancers listed in Table 5. In some embodiments, the tissue sample includes a cell including at least one of a bladder urothelial carcinoma, an acute myeloid leukemia, a brain lower grade glioma, a glioblastoma multiforme, a kidney renal clear cell carcinoma, a liver hepatocellular carcinoma, a prostate adenocarcinoma, a breast invasive carcinoma, a lung adenocarcinoma, a thyroid carcinoma, a skin cutaneous melanoma, an esophageal carcinoma, a lung squamous cell carcinoma, an uterine corpus endometrial carcinoma, a pancreatic adenocarcinoma, a stomach adenocarcinoma, a testicular germ cell tumor, an ovarian carcinoma, an ovarian serous cystadenocarcinoma, a kidney renal papillary cell carcinoma, or a high-grade serious ovarian cancer (HGSOC).

In some embodiments, the subject can be a mammalian subject. In some embodiments, the subject can be a primate. In some embodiments, the subject can be a human subject. In some embodiments, the subject can be a cancer patient.

In some embodiments, detecting the level of APOBEC3 expression can include RNA-level detection and/or quantification of APOBEC3 expression. In some embodiments, detecting the level of APOBEC3 expression includes RNA-level detection of a nucleotide sequence including an APOBEC3D RNA, an APOBEC3G RNA, or an APOBEC3H RNA.

APOBEC3 expression can be detected by any suitable method including, for example, by using quantitative reverse transcription polymerase chain reaction (RT-qPCR). In some embodiments, the level of APOBEC3 expression can be detected by, for example, Northern blotting, microarray detection, etc. In some embodiments, APOBEC3 expression can be detected via a cDNA intermediate through, for example, RTqPCR and RNAseq. In some embodiments, for example, for detecting APOBEC3G, a primer can include SEQ ID NO:1 and/or SEQ ID NO:13.

In some embodiments, detecting the level of APOBEC3 expression can include protein level detection and/or quantification. Protein expression may be determined by any suitable method including, for example, antibody-based methods and non-antibody based methods. Antibody-based methods can include, for example, western blot, flow cytometry, immunofluorescence, immunoprecipitation, enzyme-linked immunosorbent assay (ELISA), immunohistochemistry, etc. Non-antibody based methods can include, for example, protein array, interactor binding (e.g., Vif), enzyme fragment complementation, MALDI-TOF, mass spectrometry, amino acid analysis, etc.

In some embodiments, detecting the level of APOBEC3 expression can include using an anti-APOBEC3 antibody. Any suitable anti-APOBEC3 antibody can be used. The anti-APOBEC3 antibody can include, for example, an anti-APOBEC3D, an anti-APOBEC3G, or an anti-APOBEC3H antibody. In some embodiments the anti-APOBEC3 antibody can include an antibody produced by a hybridoma cell line (including those described in co-pending application number PCT/US2016/040011, which is herein incorporated by reference), or an antibody produced by phage display technologies.

In some embodiments the anti-APOBEC3 antibody can include a monoclonal antibody produced by a hybridoma cell line. In some embodiments the anti-APOBEC3 antibody can include a monoclonal antibody produced recombinantly after isolation from a hybridoma line. For example, heavy and light chains may be cloned from a hybridoma line. In some embodiments, the anti-APOBEC3 antibody includes any anti-APOBEC3D, anti-APOBEC3G, or anti-APOBEC3H antibody that works in immunohistochemistry procedures.

In some embodiments an anti-APOBEC3G antibody can include HPA001812 (Sigma-Aldrich, St. Louis, Mo.). In some embodiments the anti-APOBEC3G antibody can include a monoclonal antibody produced by hybridoma cell line 5211-110-19 (described in co-pending application number PCT/US2016/040011, which is herein incorporated by reference). In some embodiments the anti-APOBEC3G antibody can include a monoclonal antibody produced by hybridoma cell line 5210-87-13 (also described in co-pending application number PCT/US2016/040011). Although each of these rabbit monoclonal antibodies recognizes APOBEC3G, APOBEC3A, and APOBEC3B due to unavoidable homology, under certain conditions, the antibodies only detect APOBEC3G (e.g., during immunohistochemistry) because APOBEC3A is not expressed in T lymphocytes. A monoclonal antibody produced by hybridoma cell line 5211-110-19 did not recognize endogenous APOBEC3B during the immunohistochemistry procedures described herein.

In some embodiments, the method further comprises detecting an anti-APOBEC3 antibody. The anti-APOBEC3 antibody can be detected using a detection reagent including, for example, a secondary antibody, an enzyme, a fluorophore, a radioactive label, or a luminophore. In some embodiments, the anti-APOBEC3 antibody may be conjugated to a detection reagent. In some embodiments, the anti-APOBEC3 antibody may be conjugated to a chip, a biochip, a bead, a microarray, and/or other high throughput solution.

In some embodiments, the method further comprises comparing the level of expression of APOBEC3 in the sample to a control level of expression of APOBEC3. A “control level of expression” of APOBEC3 may be the level of APOBEC3 expression in non-transformed tissue of the same subject; the level of a APOBEC3 expression in tissue from a control subject; or a known value of APOBEC3 expression based on the level of expression in a tissue in a pool of control subjects. In some embodiments, a control subject may be a subject without a cancer and/or a tumor. In other embodiments, a control subject may be a subject with a similar cancer and/or tumor and a known prognosis.

In some embodiments, an increased level of APOBEC3 in the sample compared to a control level of expression of an APOBEC3 may be associated with a good prognostic outcome. For example, in some embodiments, an increased level of APOBEC3G in the sample compared to a control level of expression of APOBEC3G may be associated with a good prognostic outcome. In some embodiments, an increased level of APOBEC3D in the sample compared to a control level of expression of APOBEC3D may be associated with a good prognostic outcome. In some embodiments, an increased level of APOBEC3H in the sample compared to a control level of expression of APOBEC3H may be associated with a good prognostic outcome. In some embodiments, an increased level of a particular isoform or splice variant of APOBEC3 in a sample compared to a control level of expression of the corresponding isoform or splice variant of APOBEC3 may be associated with a good prognostic outcome. For example, an increased level of isoform 1, NP_068594.1 of APOBEC3G in the sample compared to a control level of expression of isoform 1, NP_068594.1 of APOBEC3G may be associated with a good prognostic outcome; an increased level of isoform 2, NP_001336365.1 of APOBEC3G in the sample compared to a control level of expression of isoform 2, NP_001336365.1 of APOBEC3G may be associated with a good prognostic outcome; an increased level of isoform 3, NP_001336366.1 of APOBEC3G in the sample compared to a control level of expression of isoform 3, NP_001336366.1 of APOBEC3G may be associated with a good prognostic outcome; and/or an increased level of isoform 4, NP_001336367.1 of APOBEC3G in the sample compared to a control level of expression of isoform 4, NP_001336367.1 of APOBEC3G may be associated with a good prognostic outcome.

In some embodiments, the method further comprises determining the expression of a T cell marker in the sample. In some embodiments, determining the expression of a T cell marker includes determining the level of expression of the T cell marker. In some embodiments, the T cell marker can include, for example, CD3 (delta chain isoform A, NP_000723.1; delta chain isoform B, NP_001035741.1; zeta chain isoform 1, NP_932170.1; zeta chain isoform 2, NP_000725.1; gamma chain, NP_000064.1; epsilon chain NP_000724.1), CD4 (isoform 1, NP_000607.1; isoform 2, NP_001181943.1; isoform 3, NP_001181944.1), CD8 (alpha chain isoform 1, NP_001759.3; alpha chain isoform 2, NP_741969.1; beta chain isoform 2, NP_757362.1; beta chain isoform 3, NP_742099.1; beta chain isoform 4, NP_742100.1; beta chain isoform 5, NP_004922.1; beta chain isoform 6, NP_001171571.1), GZMB (isoform 1, NP_004122.2; isoform 2, NP_001332940.1), PRF1 (NP_001076585.1), etc. In some embodiments, the T cell marker is a marker of cytotoxic T cell activation. In some embodiments determining the expression of a T cell marker comprises by detecting the transcript of a gene that encodes the T cell marker.

In some embodiments, an increased level of at least one T cell marker in the sample compared to a control level of expression of the T cell marker may be associated with a good prognostic outcome.

In some embodiments, an increased level of APOBEC3 and an increased level of at least one T cell marker in a sample compared to a control level of expression of APOBEC3 and a control level of expression of the T cell marker may be associated with a good prognostic outcome.

In some embodiments, determining the prognosis of the subject based upon the level of APOBEC3 expression can include comparing the level of expression of APOBEC3 in the sample to the level of a APOBEC3 expression in non-transformed tissue of the same subject; the level of a APOBEC3 expression in tissue from a control subject; or a known value of APOBEC3 expression based on the level of expression in a tissue in a pool of control subjects. In some embodiments, a control subject may be a subject without a cancer and/or a tumor. In other embodiments, a control subject may be a subject with a similar cancer and/or tumor and a known prognosis.

In some embodiments, determining the prognosis of the subject includes predicting a clinical outcome including, for example, the length of progression free survival (PFS) and/or overall survival (OS).

In some embodiments, determining the prognosis of the subject can include determining whether the subject is likely to be responsive to a therapy. In some embodiments, determining the prognosis of the subject can include determining the subject's responsiveness to a therapy. The therapy can include any cancer therapy alone or in combination including, for example, immunotherapy, chemotherapy, surgery, and/or radiation.

While not wishing to be bound by theory, it is believed that, in some embodiments, APOBEC3 expression is especially likely to be predictive of therapeutic success of therapies that are dependent on T cell infiltration including, for example, T cell-based immunotherapies. Because the expression of certain APOBEC3 family members (including APOBEC3D, APOBEC3G, and/or APOBEC3H) correlates with immune cell infiltration and, in particular, T cell infiltration into a tumor, the expression and/or level of expression of certain APOBEC3 family members may predict the number of T cells available to affect anti-cancer responses in the tumor. The expression and/or level of expression of certain APOBEC3 family members may further predict the number of activated or non-inhibited T cells available to affect anti-cancer responses in the tumor.

In some embodiments, the method further includes treating the patient. The treatment chosen may be based on the prognosis. The treatment may include, for example, immunotherapy, chemotherapy, surgery, and/or radiation. In some embodiments, an immunotherapy is a T cell-based immunotherapy including, for example, a chimeric antigen receptor (CAR) T cell. In some embodiments, an immunotherapy can be a checkpoint inhibitor including, for example, anti-CTLA4 and/or an anti-PD1 agent. In some embodiments, chemotherapy can include the use of a chemotherapeutic agent including, for example, carboplatin, paclitaxel, doxorubicin, gemcitabine, Topotecan, and/or a Poly (ADP-ribose) polymerase (PARP) inhibitor.

In another aspect this disclosure describes a kit for determining the level of APOBEC3 in a sample of a subject. In some embodiments the kit includes reagents for determining the level of expression of APOBEC3 in a sample and instructions for how to use the reagents. In some embodiments the kit includes instructions for how to use the reagents. In some embodiments, the instructions are FDA-approved.

In some embodiments the sample is a tissue sample. The sample can be obtained from the subject using any suitable method. For example, a biopsy can be used to obtain the sample including, for example, a punch biopsy or a core needle biopsy. In some embodiments, the sample includes a sample from a cancer. A sample from a cancer can include a tumor cell and/or a tumor infiltrating cell including, for example, a lymphocyte. In some embodiments, the sample includes a tumor cell. In some embodiments, the sample includes a tissue section.

In some embodiments, the tissue sample includes a cell including at least one of the cancers listed in Table 5. In some embodiments, the tissue sample includes a cell including at least one of a bladder urothelial carcinoma, an acute myeloid leukemia, a brain lower grade glioma, a glioblastoma multiforme, a kidney renal clear cell carcinoma, a liver hepatocellular carcinoma, a prostate adenocarcinoma, a breast invasive carcinoma, a lung adenocarcinoma, a thyroid carcinoma, a skin cutaneous melanoma, an esophageal carcinoma, a lung squamous cell carcinoma, an uterine corpus endometrial carcinoma, a pancreatic adenocarcinoma, a stomach adenocarcinoma, a testicular germ cell tumor, an ovarian carcinoma, an ovarian serous cystadenocarcinoma, a kidney renal papillary cell carcinoma, or a high-grade serious ovarian cancer (HGSOC).

In some embodiments, the subject can be a mammalian subject. In some embodiments, the subject can be a primate. In some embodiments, the subject can be a human subject. In some embodiments, the subject can be a cancer patient.

In some embodiments, the cancer is a primary tumor. In some embodiments, the cancer is metastatic (i.e., disseminated beyond the site of the primary tumor). In some embodiments, a cancer is a solid tumor. In some embodiments, the cancer is a blood cancer (for example, a leukemia or a lymphoma).

The cancer can involve any tissue or organ, such as bone, brain, breast, cervix, larynx, lung, pancreas, prostate, skin, spine, stomach, uterus, ovary, or blood. The cancer can be a bone cancer, brain cancer, breast cancer, cervical cancer, ovarian cancer, cancer of the larynx, lung cancer, pancreatic cancer, prostate cancer, skin cancer, cancer of the spine, stomach cancer, uterine cancer, or a blood cancer. The cancer can be a carcinoma. In some embodiments, the cancer includes one of the cancers listed in Table 5. In some embodiments, the cancer includes bladder urothelial carcinoma, acute myeloid leukemia, brain lower grade glioma, glioblastoma multiforme, kidney renal clear cell carcinoma, liver hepatocellular carcinoma, prostate adenocarcinoma, breast invasive carcinoma, lung adenocarcinoma, thyroid carcinoma, skin cutaneous melanoma, esophageal carcinoma, lung squamous cell carcinoma, uterine corpus endometrial carcinoma, pancreatic adenocarcinoma, stomach adenocarcinoma, testicular germ cell tumor, ovarian carcinoma, ovarian serous cystadenocarcinoma, kidney renal papillary cell carcinoma, or high-grade serious ovarian cancer (HGSOC).

In some embodiments, detecting the level of APOBEC3 expression includes detecting the level of expression of an RNA or a protein of one or more members of the APOBEC3 family. In some embodiments, detecting the level of APOBEC3 expression preferably includes detecting an RNA level of APOBEC3D, APOBEC3G, and/or APOBEC3H or detecting a protein level of APOBEC3D, APOBEC3G, and/or APOBEC3H. In some embodiments, the APOBEC3 preferably includes APOBEC3G.

In some embodiments, determining the level of expression of APOBEC3 can include RNA-level detection and/or quantification of APOBEC3 expression. In some embodiments, detecting the level of APOBEC3 expression includes RNA-level detection of a transcript including APOBEC3D, APOBEC3G, or APOBEC3H.

APOBEC3 expression can be detected by any suitable method including, for example, by using quantitative reverse transcription polymerase chain reaction (RT-qPCR). In some embodiments, the level of APOBEC3 expression can be detected by, for example, Northern blotting, microarray detection. In some embodiments, APOBEC3 expression can be detected via a cDNA intermediate through, for example, RTqPCR and RNAseq.

In some embodiments, for example, reagents for determining the level of expression of APOBEC3 G include reverse transcriptase and/or a pair of primers which are specific to separate regions of APOBEC3G. In some embodiments, the primers comprise SEQ ID NO:1 and/or SEQ ID NO:13.

In some embodiments, reagents for determining the level of expression of APOBEC3 include reagents for protein level detection and/or quantification. Protein expression may be determined by any suitable method including, for example, antibody-based methods and non-antibody based methods. Antibody-based methods can include, for example, western blot, flow cytometry, immunofluorescence, immunoprecipitation, enzyme-linked immunosorbent assay (ELISA), immunohistochemistry, etc. Non-antibody based methods can include, for example, protein array, interactor binding (e.g., Vif), enzyme fragment complementation, MALDI-TOF, mass spectrometry, amino acid analysis, etc.

In some embodiments, reagents for determining the level of expression of APOBEC3 include an anti-APOBEC3 antibody. Any suitable anti-APOBEC3 antibody can be used. The anti-APOBEC3 antibody can include, for example, an anti-APOBEC3D, an anti-APOBEC3G, or an anti-APOBEC3H antibody. In some embodiments the anti-APOBEC3 antibody can include an antibody produced by a hybridoma cell line (including those described in co-pending application number PCT/US2016/040011, which is herein incorporated by reference), or an antibody produced by phage display technologies.

In some embodiments the anti-APOBEC3 antibody can include a monoclonal antibody produced by a hybridoma cell line. In some embodiments the anti-APOBEC3 antibody can include a monoclonal antibody produced recombinantly after isolation from a hybridoma line. For example, heavy and light chains may be cloned from a hybridoma line. In some embodiments, the anti-APOBEC3 antibody includes any anti-APOBEC3D, anti-APOBEC3G, or anti-APOBEC3H antibody that works in immunohistochemistry procedures.

In some embodiments an anti-APOBEC3G antibody can include HPA001812 (Sigma-Aldrich, St. Louis, Mo.). In some embodiments the anti-APOBEC3G antibody can include a monoclonal antibody produced by hybridoma cell line 5211-110-19 (described in co-pending application number PCT/US2016/040011, which is herein incorporated by reference). In some embodiments the anti-APOBEC3G antibody can include a monoclonal antibody produced by hybridoma cell line 5210-87-13 (also described in co-pending application number PCT/US2016/040011). Although each of these rabbit monoclonal antibodies recognizes APOBEC3G, APOBEC3A, and APOBEC3B due to unavoidable homology, under certain conditions, the antibodies only detect APOBEC3G (e.g., during immunohistochemistry) because APOBEC3A is not expressed in T lymphocytes. A monoclonal antibody produced by hybridoma cell line 5211-110-19 did not recognize endogenous APOBEC3B during the immunohistochemistry procedures described herein.

In some embodiments, the reagents can include a detection reagent for detecting an anti-APOBEC3 antibody including, for example, a secondary antibody, an enzyme, a fluorophore, a radioactive label, or a luminophore. In some embodiments, the anti-APOBEC3 antibody may be conjugated to a detection reagent. In some embodiments, the anti-APOBEC3 antibody may be conjugated to a chip, a biochip, a bead, a microarray, and/or other high throughput solution.

In some embodiments, the kit may further include reagents for comparing the level of expression of APOBEC3 in the sample to a control level of expression of APOBEC3. A “control level of expression” of APOBEC3 may be the level of APOBEC3 expression in non-transformed tissue of the same subject; the level of a APOBEC3 expression in tissue from a control subject; or a known value of APOBEC3 expression based on the level of expression in a tissue from a pool of control subjects. In some embodiments, a control subject may be a subject without a cancer and/or a tumor. In other embodiments, a control subject may be a subject with a similar cancer and/or tumor and a known prognosis.

In some embodiments, the kit further includes reagents for determining the level of expression of a T cell marker. The reagents for determining the level of expression of a T cell marker can include an antibody that is specific to a T cell marker. The T cell marker can include, for example, CD3, CD3D, CD4, CD8, CD8A, GZMB, PRF1, etc. The T cell marker can be a marker of cytotoxic T cell activation. The reagents for determining the level of expression of a T cell marker can include a primer or primers that are specific to a T cell marker

In some embodiments, determining the level of APOBEC3 comprises detecting APOBEC3 expression in tumor-infiltrating T cells. APOBEC3 expression in tumor-infiltrating T cells may be detected by an suitable method including, for example, by immunohistochemistry. For example, in some embodiments, the kit may include antibodies to detect APOBEC3 and one or more T cell markers in a tissue section, and instructions to determine the co-localization of APOBEC3G with a T cell marker via immunohistochemistry. In some embodiments the kit may include antibodies to detect APOBEC3 and one or more T cell markers, and instructions to determine the co-expression of APOBEC3G with a T cell marker on a single cell via flow cytometry.

In some embodiments, the kit may further include instruction for determining the prognosis of the subject based upon the level of APOBEC3 expression. Such instructions can include comparing the level of expression of APOBEC3 in the sample to a control level of expression of APOBEC3; to the level of a APOBEC3 expression in non-transformed tissue of the same subject; to the level of a APOBEC3 expression in tissue from a control subject; or to a known value of APOBEC3 expression based on the level of expression in a tissue in a pool of control subjects.

In a further aspect this disclosure describes a method for making an anti-APOBEC3 antibody/APOBEC3 complex. In some embodiments, the method includes providing a sample comprising APOBEC3; and contacting the sample with an anti-APOBEC3 antibody under conditions that permit the binding of the anti-APOBEC3 antibody to APOBEC3 to yield the anti-APOBEC3 antibody/APOBEC3 complex. In some embodiments, the sample can be from a subject having a cancer or a tumor. In some embodiments, the sample can be from the cancer or tumor.

In some embodiments, the APOBEC3 is one or more members of the APOBEC3 family. In some embodiments, the APOBEC3 preferably includes APOBEC3D, APOBEC3G, and/or APOBEC3H. In some embodiments, the APOBEC3 preferably includes APOBEC3 G.

In some embodiments, the anti-APOBEC3 antibody includes, for example, an anti-APOBEC3D, an anti-APOBEC3G, or an anti-APOBEC3H antibody. Any suitable anti-APOBEC3 antibody can be used. In some embodiments the antibody can include an antibody produced by a hybridoma cell line (including those described in co-pending application number PCT/US2016/040011, which is herein incorporated by reference), or an antibody produced by phage display technologies.

In some embodiments an anti-APOBEC3G antibody can include HPA001812 (Sigma-Aldrich, St. Louis, Mo.). In some embodiments, the anti-APOBEC3G antibody includes a monoclonal antibody produced by hybridoma cell line 5211-110-19 (described in co-pending application number PCT/US2016/040011, which is herein incorporated by reference). In some embodiments, the anti-APOBEC3G antibody includes a monoclonal antibody produced by hybridoma cell line 5210-87-13 (described in co-pending application number PCT/US2016/040011).

The present invention is illustrated by the following examples. It is to be understood that the particular examples, materials, amounts, and procedures are to be interpreted broadly in accordance with the scope and spirit of the invention as set forth herein.

### EXAMPLE

### Materials and Methods

### Analysis of Expression Correlations in Mayo Clinic Cohort

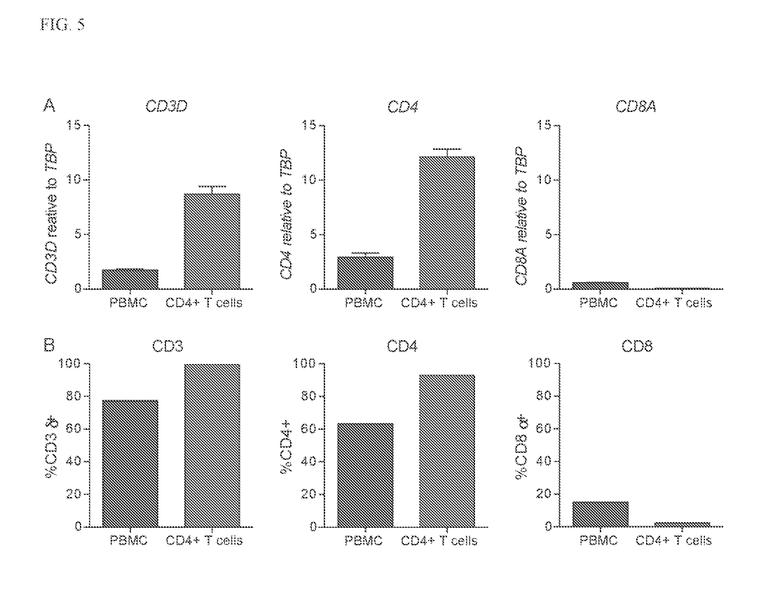

Primary tumor samples from 354 high-grade serous ovarian cancer (HGSOC) cases at the Mayo Clinic were selected based on histology, grade, stage, and availability of clinical outcome data (IRB #13-002487). Following cryosectioning of each snap frozen specimen, TRIzol based RNA extractions were performed. cDNA was synthesized in triplicate using Transcriptor Reverse Transcriptase (F. Hoffmann-La Roche, Basel, Switzerland) and reverse transcription quantitative PCR (RT-qPCR) for APOBEC3G, APOBEC3B, CD3D, CD4, CD8A, GZMB, PRF1, RNF128, and TBP was performed using the primer and probe combinations listed in Table 3 (validation in FIG. 5). Correlations between APOBEC3G, APOBEC3B, and the various T cell markers were determined using Spearman's correlation. Spearman's correlation coefficient (rₛ) and p-values are reported.

### Immunohistochemistry

Immunohistochemistry (IHC) for CD3, CD4, CD8, and APOBEC3G was performed on 7 paraffin-embedded primary HGSOC specimens obtained from patients who underwent debulking surgery at Ghent University Hospital. The tissues were fixed in a 4% formaldehyde solution for 12-48 hours and embedded in paraffin. IHC was performed on 3.5 μm tissue sections on Superfrost slides (Menzel-Glaser) using a Benchmark XT automated slide stainer (F. Hoffmann-La Roche, Basel, Switzerland), according to the manufacturer's instructions. The following antibodies were used: mouse monoclonal anti-CD3 (clone F7.2.38, dilution 1/10, Dako, Carpinteria, Calif.), rabbit monoclonal anti-CD4 (clone SP35, dilution 1/25, Cell Marque Corporation, Rocklin, Calif.), mouse monoclonal anti-CD8 (clone C8/144B, no dilution, Dako, Carpinteria, Calif.), and a rabbit monoclonal anti-APOBEC3G (clone 5211-110-19 (described in co-pending application number PCT/US2016/040011, which is herein incorporated by reference), dilution 1/50). Although this rabbit monoclonal antibody recognizes APOBEC3G, APOBEC3A, and APOBEC3B due to unavoidable homology, in studies described in this disclosure, the antibody is only detecting APOBEC3G in the immunohistochemistry because APOBEC3A is not expressed in T lymphocytes and, for reasons still under investigation, this monoclonal antibody does not recognize endogenous APOBEC3B by these procedures. APOBEC3A is not expressed in most normal cell types, and it is only induced to detectable levels in interferon-activated myeloid lineage cells (Refsland et al. Nucleic Acids Res. 2010; 38:4274-84; Koning et al. Journal of Virology. 2009; 83:9474-85; Burns et al. Nature. 2013; 494:366-70; Stenglein et al. Nat Struct. Mol. Biol. 2010; 17:222-9). Moreover, comparatively few CD68-positive macrophages were detected in the HGSOC specimens described here and positive signals from APOBEC3A or APOBEC3G expression in this cell type are minor. Heat-induced epitope retrieval was done using Cell Conditioning 1 (F. Hoffmann-La Roche, Basel, Switzerland) for CD3, CD8, and APOBEC3G, and using Cell Conditioning 2 (F. Hoffmann-La Roche, Basel, Switzerland) for CD4. Visualization of all primary antibodies was achieved with the ultraView™ Universal DAB Detection Kit (F. Hoffmann-La Roche, Basel, Switzerland). Counterstaining with hematoxylin, dehydration of the tissue sections, and application of coverslips were carried out using an automated coverslipper (Tissue-Tek, Sakura Finetek Europe B.V., the Netherlands).

### Immunofluorescent Imaging

Fluorescence-based co-localization experiments were done using a subset of the same HGSOC specimens used above for IHC following published procedures (Verschuere et al. Lab. Invest. 2011; 91:1056-67). After sample preparation, permeabilization, and blocking with 10% goat serum (X0907, Dako, Carpinteria, Calif.) in PBS at room temperature, each slide was stained first with rabbit polyclonal anti-APOBEC3G (HPA001812, Sigma-Aldrich, St. Louis, Mo.) diluted 1/50 in PBS (room temperature (RT), 1 hour), second with mouse monoclonal anti-CD3 (described above) diluted 1/10 in PBS (4° C., overnight), and finally with a combination of secondary antibodies diluted 1/500 in PBS (RT, 1 hour; anti-rabbit IgG-AlexaFluor 594 (A11012, Invitrogen, Carlsbad, Calif.) and anti-mouse IgG1-AlexaFluor 488 (A21121, Invitrogen, Carlsbad, Calif.)). Finally, slides were stained with DAPI diluted 1/5000 in methanol (RT, 5 minutes), mounted with a cover slip, and imaged using a fluorescence microscope equipped with appropriate filters (Olympus BX40, Tokyo, Japan). Multiple PBS washes were done between each step of the procedures.

### Survival Analysis in Mayo Clinic Cohort

Kaplan-Meier curves were constructed by dividing specimens at the median expression level for each gene. Overall survival (OS) data was available for all 354 patients, while progression free survival (PFS) data was only available for 348 patients. P-values, hazard ratios and 95% confidence intervals were determined using Cox regression models on the continuous log 2-transformed expression, adjusting for stage and debulking status.

### Survival Analysis in Additional Cohorts

Ovarian cancer data from The Cancer Genome Atlas (TCGA) and Gene Expression Omnibus (GEO) were compiled by the 2015 version of the Kaplan-Meier plotter database on a PostgreSQL server (Gyorffy et al. Endocr. Relat. Cancer. 2012; 19:197-208). GEO accession numbers were GSE14764, GSE15622, GSE18520, GSE19829, GSE23554, GSE26193, GSE26712, GSE27651, GSE30161, GSE3149, GSE51373, and GSE9891. All gene expression data were determined using only the HG-U133A, HG-U133 Plus 2.0, and HG-U133A 2.0 Affymetrix microarray platforms so that comparisons could be made between datasets. Specifically, APOBEC3G expression was determined using the 204205_at probe. Grade 3 serous ovarian cancers were the only samples used in this analysis. OS data were available for 856 patients, while PFS data was available for 753 patients. Kaplan-Meier plots were constructed and p-values, hazard ratios and 95% confidence intervals were calculated using the Mantel-Cox log-rank test.

Medical ethics approval for the Dutch cohort was obtained in part previously (Schuyer et al. Br. J. Cancer. 2001; 85:1359-67) (n=37) and in part more recently (n=36 and n=15; MEC-2008-183). APOBEC3G mRNA levels were measured by RTqPCR using an assay on demand (Hs00222415_m1, Applied Biosystems) and three reference genes were measured with the primers listed in Table 3 and quantification using SYBR green. Relative APOBEC3G expression levels were determined by normalization to the average of 3 reference genes as described (Sieuwerts et al. Horm. Cancer. 2014; 5:405-13). Kaplan-Meier curves were constructed by dividing specimens at the median expression level for each gene. P-values, hazard ratios and 95% confidence intervals were determined using the Mantel-Cox log-rank test.

### TCGA Analysis

Normalized RNAseqV2 data were downloaded from TCGA in July 2015. APOBEC3, CD3D, and CD20 mRNA levels were quantified using normalized read counts. rₛ and p-values for linear models of APOBEC versus immune-marker genes were calculated using Spearman's rank correlation coefficient with the R statistical environment. Cancer types were grouped by hierarchical clustering (hclust) of the rₛ values for each APOBEC family member in the R statistical environment, and these results were used to generate a dendrogram of these relationships. All data were graphed using the ggplot2 R package (Wichkham H. ggplot2: elegant graphics for data analysis. New York: Springer Publishing Company; 2016). P-values were calculated from the rₛ values and adjusted for multiple comparisons using the Benjamini-Hochberg method and significance was defined as a p-value less than 0.05.

### Results

APOBEC3G Expression Correlates with Activated T Lymphocyte Infiltration in HGSOC

APOBEC3G is expressed constitutively in many different cell lines and tissue types and is also known to be upregulated by HIV-1 infection of primary T lymphocytes, which is one of many distinct mechanisms of immune cell activation. Although virus infection is unlikely to be part of the etiology of ovarian cancer, the presence of activated immune cells (infiltrate) in HGSOC is known to correlate with better overall outcomes, most likely due to strong anti-tumor immune responses (Knutson et al. Cancer Immunol Immunother. 2015; 64:1495-504; et al. Clin Cancer Res. 2012; 18:3281-92; Zhang et al. N Engl J Med. 2003; 348:203-13; Sato et al. Proc Natl Acad Sci USA. 2005; 102:18538-43; Preston et al. PLoS ONE. 2013; 8:e80063). To determine whether APOBEC3G expression correlated with T cell infiltration and clinical outcomes, a cohort of 354 primary HGSOC samples procured at the Mayo Clinic (clinical characteristics in Table 1) were used.

TABLE 1
Clinical information for Mayo cohort (n = 354)

| Tumor characteristic | Number |
|---|---|
| Morphology | |
| Serous | 354 (100%) |
| Grade | |
| 2 | 11 (3.1%) |
| 3 | 343 (96.9%) |
| Stage | |
| 1 | 14 (3.9%) |
| 2 | 8 (2.3%) |
| 3 | 253 (71.5%) |
| 4 | 79 (22.3%) |
| Debulking status | |
| No residual disease | 162 (45.8%) |
| <=1 cm remaining | 144 (40.7%) |
| <=1 cm remaining, possibly 0 | 48 (13.5%) |

RNA was prepared from fresh frozen HGSOC tissues, and a previously validated, highly specific RT-qPCR assay was used to assess APOBEC3G mRNA levels (Refsland et al. Nucleic Acids Res. 2010; 38:4274-84). In parallel, expression of the related gene APOBEC3B, which has been implicated in ovarian cancer genome mutagenesis (Leonard et al. Cancer Research. 2013; 73:7222-31), was assayed. In addition, the mRNA levels of several established T cell markers were quantified, including CD3D (total T cells), CD4 (helper T cells), CD8A (cytotoxic T cells), GZMB (activated cytotoxic T cells), PRF1 (activated cytotoxic T cells), and RNF128 (anergic T cells) (primers and probes in Table 4) (Sabek et al. Transplantation. 2002; 74:701-7; Zheng et al. EMBO Rep. 2008; 9:50-5).

Strong positive correlations were observed between APOBEC3G mRNA expression levels and CD3D (p<0.0001, rₛ=0.6159), CD4 (p<0.0001, rₛ=0.5825), CD8A (p<0.0001, rₛ=0.6168), GZMB (p<0.0001, rₛ=0.6591), and PRF1 (p<0.0001, rₛ=0.6422) (FIG. 1A-E). The lone exception was RNF128 (p=0.7665, rₛ=0.0161), which is a marker for T cell anergy, suggesting that the positive signals emanate from bona fide activated T lymphocytes (FIG. 1F). Interestingly, APOBEC3G expression showed a similar positive correlation with CD8A and CD4, suggesting that APOBEC3G is expressed in both the cytotoxic and helper T cell subsets (FIG. 1B vs. 1C). This result was corroborated by similarly strong positive correlations between APOBEC3G and two markers of CTL activation, GZMB and PRF1 (FIGS. 1D and 1E). In contrast, weaker and less significant correlations were found between APOBEC3B and the expression of any of these T cell genes (FIG. 1G-L). This result was expected, however, because prior studies have indicated that APOBEC3B is only expressed at low levels in normal tissues and upregulated in tumor cells. Taken together, these data confirm T cell infiltration in HGSOC and reveal an unanticipated association between high levels of APOBEC3G expression and CTL activation in HGSOC. This result for APOBEC3G was especially unexpected given its previously documented broad expression profile (Refsland et al. Nucleic Acids Res. 2010; 38:4274-84; Koning et al. Journal of Virology. 2009; 83:9474-85; Liddament et al. Curr Biol. 2004; 14:1385-91; Burns et al. Nature. 2013; 494:366-70).

### APOBEC3G Protein Visualization in Infiltrating T Lymphocytes in HGSOC

To determine whether APOBEC3G protein expression co-localizes with the same T cell markers analyzed above at the mRNA level by RT-qPCR, hematoxylin staining and immunohistochemistry were performed for CD3, CD4, CD8, and APOBEC3G on seven unrelated HGSOC samples (representative image sets in FIG. 2A-T with hematoxylin staining in FIGS. 2A, F, K, and P). As expected from the RT-qPCR analysis above, several of these additional tumor specimens showed clear evidence for T lymphocyte infiltration. Two HGSOC lesions contained low expression of all examined T cell markers (representative images for CD3, CD4, and CD8 in FIG. 2B-D) and correspondingly low levels of APOBEC3G expression (representative image in FIG. 2E). In contrast, two tumors showed extensive T lymphocyte infiltration (representative images for CD3, CD4 and CD8 in FIGS. 2G-2I, 2L-2N, and 2Q-2S). Interestingly, there was a strong colocalization between these markers and the expression of APOBEC3G in these tumors (representative image in FIGS. 2J, 2O, and 2T). Moreover, different regions of a single HGSOC can be heterogenous, with one region showing dense clusters of APOBEC3G-high infiltrating T cells and another showing a more dispersed distribution (compare FIGS. 2F-J and 2K-O). The remaining three samples showed moderate expression of both infiltrating T cell markers and APOBEC3G, and colocalization was still observed. These immunohistochemical experiments confirm the above correlations and show that APOBEC3G is indeed expressed at the protein level within tumor infiltrating T lymphocytes.

To directly test whether APOBEC3G is expressed within tumor infiltrating T lymphocytes, a series of fluorescence-based co-localization experiments with CD3 and APOBEC3G were performed. Although groups of cells caused excessive background fluorescence, images of well-isolated infiltrating T lymphocytes demonstrate for the first time that CD3 and APOBEC3G are indeed co-expressed in the same T lymphocyte (representative image in FIG. 2U-2X). Moreover, the DAPI co-stain and 1000× total magnification combine to show that both proteins are excluded from the nuclear compartment and predominantly cytoplasmic (the additional cell surface localization of CD3 is more difficult to visualize in tissue cross sections).

### APOBEC3G is a Candidate Biomarker for Improved HGSOC Patient Outcomes

Long-term clinical follow-up data were available for all of the Mayo Clinic HGSOC patients. The aforementioned gene expression data were therefore correlated with clinical information to determine whether APOBEC3G expression levels predict the length of progression free survival (PFS) and/or overall survival (OS) in HGSOC. As a positive control, the T cell markers above were also analyzed with respect to clinical information. Kaplan-Meier plots were constructed by splitting each gene expression data set at the median to create high and low expression groups (FIGS. 3A and 3B). P-values, hazard ratios (HR), and 95% confidence intervals (CI) were determined using Cox regression models on the log 2-transformed expression that were corrected for stage and debulking status (Table 2).

TABLE 2
Mayo cohort Cox regression analysis

| Gene | Parameter¹ | p-value | HR² | 95% CI³ |
|---|---|---|---|---|
| CD3D | PFS | 0.020 | 0.94 | [0.90, 0.99] |
| CD3D | OS | 0.087 | 0.95 | [0.90, 1.01] |
| CD4 | PFS | 0.0046 | 0.90 | [0.84, 0.97] |
| CD4 | OS | 0.018 | 0.91 | [0.84, 0.98] |
| CD8A | PFS | 0.0053 | 0.93 | [0.88, 0.98] |
| CD8A | OS | 0.015 | 0.93 | [0.87, 0.99] |
| GZMB | PFS | 0.011 | 0.94 | [0.90, 0.99] |
| GZMB | OS | 0.047 | 0.95 | [0.90, 1.00] |
| PRF1 | PFS | 0.0049 | 0.91 | [0.86, 0.97] |
| PRF1 | OS | 0.018 | 0.92 | [0.86, 0.99] |
| RNF128 | PFS | 0.43 | 0.97 | [0.91, 1.04] |
| RNF128 | OS | 0.44 | 0.97 | [0.91, 1.04] |
| APOBEC3G | PFS | <0.0001 | 0.81 | [0.73, 0.89] |
| APOBEC3G | OS | 0.0003 | 0.82 | [0.73, 0.91] |
| APOBEC3B | PFS | 0.034 | 0.92 | [0.85, 0.99] |
| APOBEC3B | OS | 0.06 | 0.93 | [0.87, 1.00] |

¹OS = overall survival (n = 354); PFS = progression free survival (n = 348)
²HR = hazard ratio
³CI = confidence interval

Higher expression levels of CD3D (p=0.020, HR=0.94 [95% confidence interval 0.90, 0.99]), CD4 (p=0.0046, HR=0.90 [0.84, 0.97]), CD8A (p=0.0053, HR=0.93 [0.88, 0.98]), GZMB (p=0.011, HR=0.94 [0.90, 0.99]), and PRF1 (p=0.0049, HR=0.91 [0.86, 0.97]) were all associated with improved PFS (FIG. 3A and Table 2). As expected, RNF128 (p=0.43, HR=0.97 [0.91, 1.04]) did not correlate with PFS (FIG. 3A and Table 2). Interestingly, APOBEC3G (p<0.0001, HR=0.81 [0.73, 0.89]) surpassed all of these genes as the most indicative marker of improved PFS in HGSOC (FIG. 3A). The results compiled from an analysis of OS largely mirrored those of PFS (FIG. 3B and Table 2). Next, Kaplan-Meier plotter (kmplot.com) was used to generate a validation cohort using HGSOC data from TCGA and GEO. In this composite analysis of HGSOC patients, high levels of APOBEC3G correlated with improved durations of PFS (p=0.0057, HR=0.78 [0.65, 0.93]) and OS (p=0.063, HR=0.84 [0.70, 1.01]) (FIG. 6). Although only the former correlation reached statistical significance, the latter trended toward significance and also supported the observations above with the HGSOC Mayo Clinic cohort. A larger degree of variation is to be expected in this composite cohort because clinical variables are more difficult to take into account.

Finally, HGSOC specimens from a Dutch cohort consisting of 88 patient samples (clinical information in Table 4) were analyzed. APOBEC3G expression was quantified using an independent RT-qPCR strategy (housekeeping gene primer sequences in Table 4 and see Materials and Methods for details). Again, APOBEC3G expression levels associated with improved OS and more weakly with PFS (FIG. 6).

APOBEC3B Expression Levels do not Associate with HGSOC Outcomes

APOBEC3B has been implicated recently as an endogenous mutagen in several cancers, including ovarian cancer. Moreover, its overexpression has been linked to poor patient outcomes in multiple cancer types. Using the Mayo cohort gene expression data and clinical information from above, the effect of APOBEC3B on patient outcomes in HGSOC was examined. In contrast to prior reports for other cancers, the Mayo cohort showed a trend toward high APOBEC3B and improved, rather than worsened, PFS and OS outcomes, although these relationships were less significant statistically that those for APOBEC3G (FIG. 3, Table 2).

APOBEC Expression Correlates with Immune Cell Markers in Multiple Human Cancers

To extend the findings from HGSOC to additional human cancers, publically available RNAseq data from TCGA were analyzed to determine if correlations exist between expression of APOBEC genes and immune cell markers. At the time of these analyses, the TCGA had RNAseq data available for 7,861 samples spanning 22 different tumor types (details in Table 5). For each tumor type, expression of each APOBEC family member was quantified and correlated with the T cell marker CD3D (FIG. 4 top heatmap). Hierarchical clustering was also performed to elucidate similar correlation patterns between cancer types (FIG. 4). These analyses revealed that, in addition to APOBEC3G, APOBEC3D and APOBEC3H also correlated significantly with CD3D across multiple cancer types. APOBEC3F, also known to be expressed broadly and in T cells, did not correlate as strongly. The same analysis was also performed with CD20, a well-known marker for B cells (FIG. 4 bottom heatmap). The expression of the antibody diversification gene, AID, was the only APOBEC family member that significantly correlated with CD20 in a majority of cancer types (FIG. 4). These analyses indicate that much of the expression of several APOBEC family members in cancer is likely due to T and B cell infiltration.

TABLE 3
RT-qPCR primer and probe sets

| | | | | |
|---|---|---|---|---|
| Gene | mRNA NCBI | | | |
| symbol | accession | 5′ Primer sequence | 3′ Primer sequence | Probe |

Mayo Clinic cohort analysis

| | | | | |
|---|---|---|---|---|
| APOBEC3G | NM_021822 | ccgaggacccgaaggttac | tccaacagtgctgaaattcg | UPL79 |
| | | (SEQ ID NO: 1) | (SEQ ID NO: 13) | |
| APOBEC3B | NM_004900 | gaccctttggtccttcgac | gcacagccccaggagaag | UPL1 |
| | | (SEQ ID NO: 2) | (SEQ ID NO: 14) | |
| CD3D | NM_000732 | ctaccgtgcaagttcattatcg | aaggagcagagtggcaatga | UPL83 |
| | | (SEQ ID NO: 3) | (SEQ ID NO: 15) | |
| CD4 | NM_000616 | gatacttacatctgtgaagtggagga | agcaggtgggtgtcagagtt | UPL63 |
| | | (SEQ ID NO: 4) | (SEQ ID NO: 16) | |
| CD8A | NM_001768 | tcatggccttaccagtgacc | aggttccaggtccgatcc | UPL51 |
| | | (SEQ ID NO: 5) | (SEQ ID NO: 17) | |
| GZMB | NM_004131 | gagacgacttcgtgctgaca | ccccaaggtgacatttatgg | UPL60 |
| | | (SEQ ID NO: 6) | (SEQ ID NO: 18) | |
| PRF1 | NM_001083116 | ccgcttctctatacgggattc | gcagcagcaggagaaggat | UPL68 |
| | | (SEQ ID NO: 7) | (SEQ ID NO: 19) | |
| RNF128 | NM_024539 | gtgcacctcttgccttacg | ccttttatttcacaacgacagaaa | UPL51 |
| | | (SEQ ID NO: 8) | (SEQ ID NO: 20) | |
| TBP | NM_003194 | cccatgactcccatgacc | tttacaaccaagattcactgtgg | UPL51 |
| | | (SEQ ID NO: 9) | (SEQ ID NO: 21) | |

Dutch cohort analysis

| | | | | |
|---|---|---|---|---|
| APOBEC3G | NM_021822 | * | * | * |
| HMBS | NM_000190 | catgtctggtaacggcaatg | gtacgaggctttcaatgttg | N/A |
| | | (SEQ ID NO: 10) | (SEQ ID NO: 22) | |
| HPRT1 | NM_000194 | tattgtaatgaccagtcaacag | ggtccttttcaccagcaag | N/A |
| | | (SEQ ID NO: 11) | (SEQ ID NO: 23) | |
| TBP | NM_003194 | ttcggagagttctgggattg | acgaagtgcaatggtctttag | N/A |
| | | (SEQ ID NO: 12) | (SEQ ID NO: 24) | |

*This primer/probe set is from Applied Biosystems TagMan Gene Expression Assay Hs00222415_m1, and the corresponding DNA sequences are proprietary information. Assay validation was reported previously (32).

TABLE 4
Clinical information for Dutch cohort (n = 88)

| Tumor characteristic | Number |
|---|---|
| Morphology | |
| Serous | 77 (87.5%) |
| Undifferentiated | 11 (12.5%) |
| Grade | |
| 2 | 34 (38.6%) |
| 3 | 54 (61.4%) |
| Stage | |
| 1 | 10 (11.4%) |
| 2 | 3 (3.4%) |
| 3 | 54 (61.4%) |
| 4 | 18 (20.5%) |
| Unknown | 3 (3.4%) |
| Debulking status | |
| No residual disease | 14 (15.9%) |
| <=1 cm remaining | 24 (27.3%) |
| <=1 cm remaining, possibly 0 | — |
| ‘>1 cm | 27 (30.7%) |
| Unknown | 23 (26.1%) |

TABLE 5
Summary of samples used in TCGA analysis

| | TCGA | Number |
|---|---|---|
| Cancer type | abbreviation | of samples |
| Bladder urothelial carcinoma | BLCA | 408 |
| | BRCA | 1,066 |
| Cervical squamous cell carcinoma and | CESC | 306 |
| endocervical adenocarcinoma | | |
| Esophageal carcinoma | ESCA | 185 |
| Glioblastoma multiforme | GBM | 169 |
| Head and neck squamous cell carcinoma | HNSC | 521 |
| Kidney renal clear cell carcinoma | KIRC | 103 |
| Kidney renal papillary cell carcinoma | KIRP | 291 |
| Acute Myeloid Leukemia | LAML | 173 |
| Brain lower grade glioma | LGG | 534 |
| Liver hepatocellular carcinoma | LIHC | 327 |
| Lung adenocarcinoma | LUAD | 513 |
| Lung squamous cell carcinoma | LUSC | 502 |
| Ovarian serous cystadenocarcinoma | OV | 266 |
| Pancreatic adenocarcinoma | PAAD | 99 |
| Prostate adenocarcinoma | PRAD | 498 |
| Rectum adenocarcinoma | READ | 167 |
| Skin cutaneous melanoma | SKCM | 472 |
| Stomach adenocarcinoma | STAD | 415 |
| Testicular germ cell tumors | TGCT | 156 |
| Thyroid carcinoma | THCA | 513 |
| Uterine corpus endometrial carcinoma | UCEC | 177 |
| | Total | 7,861 |

All headings are for the convenience of the reader and should not be used to limit the meaning of the text that follows the heading, unless so specified.

The complete disclosure of all patents, patent applications, and publications, and electronically available material (including, for instance, nucleotide sequence submissions in, e.g., GenBank and RefSeq, and amino acid sequence submissions in, e.g., SwissProt, PIR, PRF, PDB, and translations from annotated coding regions in GenBank and RefSeq) cited herein are incorporated by reference. In the event that any inconsistency exists between the disclosure of the present application and the disclosure(s) of any document incorporated herein by reference, the disclosure of the present application shall govern. The foregoing detailed description and examples have been given for clarity of understanding only. No unnecessary limitations are to be understood therefrom. The invention is not limited to the exact details shown and described, for variations obvious to one skilled in the art will be included within the invention defined by the claims.

Unless otherwise indicated, all numbers expressing quantities of components, molecular weights, and so forth used in the specification and claims are to be understood as being modified in all instances by the term “about.” Accordingly, unless otherwise indicated to the contrary, the numerical parameters set forth in the specification and claims are approximations that may vary depending upon the desired properties sought to be obtained by the present invention. At the very least, and not as an attempt to limit the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the invention are approximations, the numerical values set forth in the specific examples are reported as precisely as possible. All numerical values, however, inherently contain a range necessarily resulting from the standard deviation found in their respective testing measurements.

1. A method comprising:
detecting a level of APOBEC3 expression in a sample from a subject; and
determining a prognosis of the subject based upon the level of APOBEC3 expression.

2.-67. (canceled)


SEQUENCE LISTING (24 sequences)

SEQ ID NO: 1 - DNA, 19 bp, Artificial
  Primer
ccgaggacccgaaggttac

SEQ ID NO: 2 - DNA, 19 bp, Artificial
  Primer
gaccctttggtccttcgac

SEQ ID NO: 3 - DNA, 22 bp, Artificial
  Primer
ctaccgtgcaagttcattatcg

SEQ ID NO: 4 - DNA, 26 bp, Artificial
  Primer
gatacttacatctgtgaagtggagga

SEQ ID NO: 5 - DNA, 20 bp, Artificial
  Primer
tcatggccttaccagtgacc

SEQ ID NO: 6 - DNA, 20 bp, Artificial
  Primer
gagacgacttcgtgctgaca

SEQ ID NO: 7 - DNA, 21 bp, Artificial
  Primer
ccgcttctctatacgggattc

SEQ ID NO: 8 - DNA, 19 bp, Artificial
  Primer
gtgcacctcttgccttacg

SEQ ID NO: 9 - DNA, 18 bp, Artificial
  Primer
cccatgactcccatgacc

SEQ ID NO: 10 - DNA, 20 bp, Artificial
  Primer
catgtctggtaacggcaatg

SEQ ID NO: 11 - DNA, 22 bp, Artificial
  Primer
tattgtaatgaccagtcaacag

SEQ ID NO: 12 - DNA, 20 bp, Artificial
  Primer
ttcggagagttctgggattg

SEQ ID NO: 13 - DNA, 20 bp, Artificial
  Primer
tccaacagtgctgaaattcg

SEQ ID NO: 14 - DNA, 18 bp, Artificial
  Primer
gcacagccccaggagaag

SEQ ID NO: 15 - DNA, 20 bp, Artificial
  Primer
aaggagcagagtggcaatga

SEQ ID NO: 16 - DNA, 20 bp, Artificial
  Primer
agcaggtgggtgtcagagtt

SEQ ID NO: 17 - DNA, 18 bp, Artificial
  Primer
aggttccaggtccgatcc

SEQ ID NO: 18 - DNA, 20 bp, Artificial
  Primer
ccccaaggtgacatttatgg

SEQ ID NO: 19 - DNA, 19 bp, Artificial
  Primer
gcagcagcaggagaaggat

SEQ ID NO: 20 - DNA, 24 bp, Artificial
  Primer
ccttttatttcacaacgacagaaa

SEQ ID NO: 21 - DNA, 23 bp, Artificial
  Primer
tttacaaccaagattcactgtgg

SEQ ID NO: 22 - DNA, 20 bp, Artificial
  Primer
gtacgaggctttcaatgttg

SEQ ID NO: 23 - DNA, 19 bp, Artificial
  Primer
ggtccttttcaccagcaag

SEQ ID NO: 24 - DNA, 21 bp, Artificial
  Primer
acgaagtgcaatggtctttag